(19) United States
(12) Patent Application Publication
(10) Pub. No.: US 2013/0005027 A1
(43) Pub. Date: Jan. 3, 2013

(54) FILTRATION DEVICE AND SYSTEM

(76) Inventors: Patrick Paullier, Thourotte (FR); Aissa Ould Dris, Compiegne (FR); Eric Leclerc, Margny Les Compiegne (FR)

(21) Appl. No.: 13/497,247

(22) PCT Filed: Sep. 23, 2010
(86) PCT No.: PCT/EP10/64082

§ 371 Date: Sep. 21, 2012

(30) Foreign Application Priority Data
Sep. 23, 2009 (FR) 0956568

(51) Int. Cl. C12M 1/12

(52) U.S. Cl. 435/297.1

(57) ABSTRACT

The invention relates to a filtration device (100), characterized in that it includes: a first block (101) having a cavity forming a first chamber (110) comprising a bottom wall (111) having a set of microstructures including micro-walls and micro-contacts, the set of microstructures defining micro-chambers and micro-channels on the bottom wall (111) of the culture chamber; a second block (102) having a cavity forming a second chamber (120); and a filtration membrane (130), the first block (101), the membrane (130), and the second block (102) being arranged such that the membrane (130) is located between the first chamber (110) and the second chamber (120), adjacent to each of the first and second chambers (110, 120); as well as a first opening and a second opening for enabling a first fluid to pass into the second chamber (120) which is separated from the first chamber (110) by the membrane (130).

### GENERAL TECHNICAL FIELD

The present invention relates to reproduction in vitro of filtration phenomena.

More specifically, it relates to a filtration device for a bioreactor with a membrane separating two chambers and a filtration system implementing one or more examples of such a filtration device.

### STATE OF THE ART

Transplants presently remain the most efficient solution for treating liver disorders and kidney dysfunctions. However, insufficiency of donors forces patients awaiting an organ to undergo major and regular treatments most often sources of complications. One of the great challenges of tissue engineering therefore lies in the development of artificial organs capable of replacing failing or absent organs. The patients would then see their life conditions improve and the costs of the treatments decrease. For this purpose, many investigations have been conducted in order to reproduce in vitro phenomena internal to the organs of the human and animal body, in particular filtration phenomena.

To do this, it is necessary to have available bioreactors reproducing an environment favorable to the development and organization of cells, close to that of a animal or human tissue or organ on the one hand, membranes capable of reproducing the filtration phenomena for example, glomerular filtration in kidneys. Many solutions have been proposed in the prior art.

Document U.S. Pat. No. 6,197,575 presents a device for cultivating cells in order to obtain artificial tissues or organs in vitro. This device comprises an enclosure with a filtration membrane separating the enclosure into two chambers, a membrane on which are arranged channels in order to receive a cell culture. Thus, the membrane playing the role of a filter is also used as a support for cultivating cells in a culture chamber, the other portion of the enclosure forming a discharge chamber. This suggests that the membrane has some rigidity. Various fluid inflow/outflow combinations for culture and discharge chambers are contemplated (see FIGS. 1 to 2c of document U.S. Pat. No. 6,197,575), each combination corresponding to a specific use of the device.

The article <<A MEMS-Based Renal Replacement System>> published in June 2004 describes a unit for treating blood provided for application in continuous hemodialysis. The unit consists of a stack of bilayer devices each comprising a network of channels for blood circulation and a discharge chamber, both networks facing each other and separated by an ultrafiltration membrane (see FIG. 2 of this document). The authors of this article used an algorithm for defining the morphology of a network reproducing blood circulation conditions in human blood vessels.

If the presented unit gives the possibility of effectively mimicking the blood transport conditions in blood vessels, it is not without posing problems. Indeed, the blood is confined in channels with a very narrow section (a height of 35 microns) which limits the blood flow rate treated by each device of the entity and may induce clogging problems of the channels. The authors foresee not less than 100 bilayer devices required for carrying out hemodialysis.

U.S. Pat. No. 7,048,856 presents a compact ultrafiltration device which may be used as a bioreactor. The device comprises a chamber in which is placed an ultrafiltration membrane, said membrane separates the chamber into a filtration portion and a discharge portion, as well as a fluid inlet, a filtration fluid outlet and a fluid discharge outlet. The membrane is adapted to the fluid at the inlet and to the contemplated filtration. For example, the membrane may have pores, the size of which is selected in order to filter urea in the blood. Moreover, according to a particular embodiment shown in this document, the device may comprise an analysis chamber in which the filtered fluid is analyzed. The membrane may also receive a cell culture. The fluid inlet and outlet may be provided with pumps or valves for controlling the flow rate, optionally connected to pressure sensors.

Document WO 2004/020590 describes a bioreactor intended for cultivating living cells, in which the conditions of the human body are reproduced artificially. In order to access better understanding of certain dysfunctions of the mechanisms of the body, it is necessary to develop bioreactors capable of mimicking the micro-environment of abnormal tissues in vitro. An application proposed by the document describes a chamber divided into two sub-chambers containing cells of a first type and cells of a second type respectively. Both sub-chambers are separated by a porous barrier which may be totally impervious or else pervious to certain specific cells. The bioreactor also comprises inlet/outlet accesses allowing circulation of cells, fluids or chemicals in each of the sub-chambers. By adding various substrates positioned in a suitable way in the bioreactor, it is possible to proceed with electrochemical and optical measurements.

Thus, a large variety of in vitro filtration devices have been proposed in the past.

### PRESENTATION OF THE INVENTION

The invention proposes a filtration of a novel type having many advantages as compared with the solutions proposed in the prior art.

For this purpose, the invention proposes according to a first aspect, a filtration device characterized in that it comprises:
a first block having a cavity forming a first chamber including a bottom wall having a set of microstructures comprising microwalls and microbumps, the set of microstructures defining on the bottom wall of the culture chamber, microchambers and microchannels,
a second block having a cavity forming a second chamber, and
a filtration membrane,
the first block, the membrane and the second block being laid out so that the membrane is located between the first chamber and the second chamber, adjacent to each of the first and second chambers,
a first opening and a second opening for letting through a fluid into the second chamber separated from the first chamber by the membrane.

The filtration device according to the first aspect of the invention is advantageously completed by the following features, taken alone or in any of their technically possible combinations:
a fluid inlet in the first chamber connected to at least one portion of the microchannels and a fluid outlet in the first chamber connected to at least one portion of the microchannels are included, the microchannels forming a network connecting the fluid inlet to each microchamber and each microchamber to the fluid outlet, so as to allow fluid circulation in the microchambers of the first chamber,
the microchambers have a length dimension and a width dimension relatively to a circulation direction of the fluid, each comprised between 500 μm and 550 μm,
the microchannels have a length dimension relative to a circulation direction of the fluid comprised between 700 μm and 750 μm and a width dimension relatively to a circulation direction of the fluid comprised between 200 μm and 250 μm,
the microchambers comprise an inlet area and an outlet area, the microwalls comprising angled areas on either side of the inlet area and of the outlet area of at least one chamber, the angled areas having a width dimension relative to a circulation direction of the fluid comprised between 100 μm and 120 μm so as to define on either side of the inlet area of said chamber, partly protected areas,
the first chamber has a culture surface area in a ratio with an overall surface area of the bottom wall, comprised between 90% and 110%,
the first chamber has a culture surface area in a ratio with an overall available volume of fluid of the first chamber, comprised between 4/mm and 6/mm,
the second chamber comprises an upper wall having a set of microstructures identical with that of the bottom wall of the first chamber,
the membrane is in a flexible material,
the membrane is in a hydrophilic material,
the membrane is in a hydrophobic material,
the membrane is a barrier membrane,
the membrane is in a suitable material for allowing cells to be grown on the membrane, and
a holding means having a locked configuration in which the first block and the membrane are firmly held together on the one hand, the membrane and the second block are firmly held together on the other hand, and an unlocked configuration, in which the block and the membrane may be separated from each other and/or in which the membrane and the second block may be separated from each other, the holding means being able to be switched from the locked configuration to the unlocked configuration and vice versa, is included.

The invention also proposes, according to a second aspect, a filtration system, characterized in that it comprises:
a filtration device according to the first aspect of the invention, and
a fluid circuit comprising circulation piping provided with a circulation means and connected to the first and second openings in the second chamber.

The filtration system according to the second aspect of the invention is advantageously completed by the following features, taken alone or in any of their technically possible combinations:
a fluid inlet in the first chamber connected to at least one portion of the microchannels and a fluid outlet in the first chamber connected to at least one portion of the microchannels are included, the microchannels forming a network connecting the fluid inlet to each microchamber and each microchamber to the fluid outlet, so as to allow fluid circulation in the microchambers of the first chamber, the device further comprising a fluid circuit comprising circulation piping provided with a circulation means and connected to the fluid inlet and outlet in the first chamber,
the first circuit comprises a control means for controlling fluid pressure in the first chamber, and
the second circuit comprises a control means for controlling fluid pressure in the second chamber.

According to a third aspect, the invention proposes a filtration system comprising several filtration devices according to the first aspect of the invention connected through circulation circuits.

### PRESENTATION OF THE FIGURES

Other features, objects and advantages of the invention will become apparent from the following description, which is purely illustrative and non-limiting, and which should be read with reference to the appended drawings wherein:

FIG. 1 schematically illustrates a filtration device in a front sectional view according to a possible embodiment of the first aspect of the invention,

FIG. 2 schematically illustrates a filtration device in a side sectional view according to a possible embodiment of the first aspect of the invention,

FIG. 3 illustrates a three-dimensional perspective view of microstructures according to a possible embodiment of the first aspect of the invention,

FIG. 4 schematically illustrates a filtration system in a side sectional view according to a possible embodiment of the second aspect of the invention,

FIG. 5 illustrates a filtration device in a side sectional view according to a possible embodiment of the first aspect of the invention in which the first chamber is provided with a fluid inlet and outlet,

FIG. 6 schematically illustrates a filtration device in a top sectional view according to a possible embodiment of the first aspect of the invention in which the first chamber is provided with a fluid inlet network and outlet network,

FIG. 7 schematically illustrates in a top view, microstructures of the bottom wall of the first chamber, as well as dimensions of these microstructures according to a possible embodiment of the first aspect of the invention,

FIGS. 8a and 8b illustrate electron microscopy images of membranes of different porosities according to possible embodiments of the first aspect of the invention,

FIGS. 9 and 10 schematically illustrate a filtration system in a side sectional view according to possible embodiments of the second aspect of the invention,

FIGS. 11 and 12 graphically illustrate a time-dependent change in the concentration-over-initial-concentration ratio in the second chamber during an experiment using a system according to a possible embodiment of the second aspect of the invention,

FIG. 13 schematically illustrates a filtration system in a side sectional view according to possible embodiments of the second aspect of the invention, and

FIG. 14 schematically illustrates an experiment for characterizing the water slope of a membrane, which experiment applies a system according to a possible embodiment of the second aspect of the invention.

In the different figures, similar elements bear the same numerical references.

### DETAILED DESCRIPTION

### General Presentation

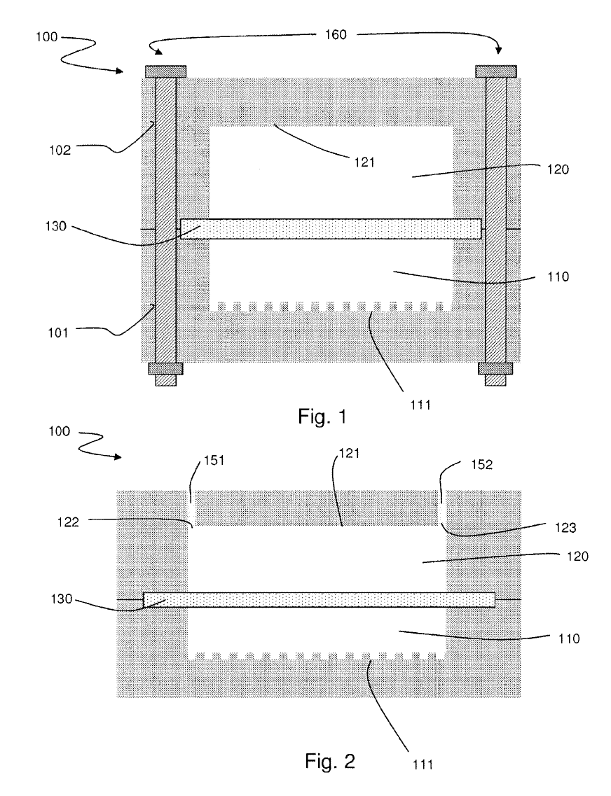

With reference to FIGS. 1 and 2, a filtration device 100 according to the first aspect of the invention comprises a first block 101 having a cavity which forms a first chamber 110, a second block 102 also having a cavity which forms a second chamber 102, as well as a filtration membrane 130 positioned between the first chamber 110 and the second chamber 120 and adjacent to each of the first and second chambers 110, 120.

Both of these chambers may receive a fluid to be filtered or an operating fluid. As this will be described later on, the membrane 130 allows transport of material from one fluid to the other by a concentration difference or else by a pressure difference between the first and the second chamber, or further by any other filtration cause known to one skilled in the art.

As a non-limiting example, the first and second blocks 101, 102 may consist of glass, silica or advantageously polymers such as polymethyl methacrylate (PMMA) or polydimethylsiloxane (PDMS) or a mixture thereof. PDMS has the advantage of being porous to oxygen. Both blocks may consist of the same material or else be in different materials.

The second chamber 120 is intended to receive a circulating fluid. For this purpose, the device 100 comprises a first opening 122 and a second opening 123 for letting through a fluid. It further has an upper wall 121.

By <<membrane>> is meant a wall separating two media. A membrane has a porosity depending on the size of its pores. In this case, according to the first aspect of the invention, the membrane separates the first chamber 110 and the second chamber 120 and defines a single closed space in complementarity with each of these chambers 110, 120. In particular, in the absence of a pressure difference between both chambers 110, 120, the membrane has no contact point with the bottom wall 111 of the first chamber 110, nor with the upper wall 121 of the second chamber 120.

As this will be described in detail later on, the first opening 122 (the second opening 123 respectively) may be a fluid inlet in the second chamber 120 (a fluid outlet of the second chamber 120, respectively) or else a fluid outlet of the second chamber 120 (a fluid inlet in the second chamber 120, respectively) depending on the direction of the flow provided in chamber 120.

The first chamber 110 has a bottom wall 111 preferentially having a substantially rectangular shape which defines a length and a width.

As a non limiting example, the length and the width of the bottom wall may measure 13.25 mm and 11.23 mm, respectively.

In the following, the horizontal is defined as being parallel to the bottom wall 111 and the vertical as orthogonal to the wall 111 and directed from the wall 111 towards the membrane 130. Thus, it will be stated that the bottom wall 111 is horizontal and located below the membrane 130, itself located below the upper wall 121. Moreover, the longitudinal direction of the bottom wall 111 will be noted as d1.

These definitions have the purpose of clarifying the remainder of the text and should by no means be interpreted as a limitation of the position of use of the device according to the first aspect of the invention in any co-ordinate system.

### Microstructures for Cultivating Cells

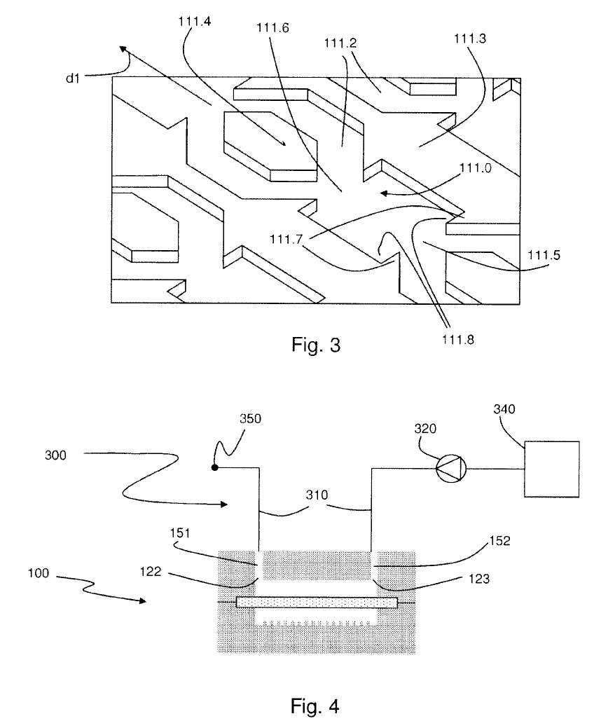

The first chamber 110 is intended to receive a cell culture. For this purpose, the bottom wall 111 has a set of microstructures illustrated in three dimensions in FIG. 3. These microstructures were developed by the applicant and have already been the subject, in their form, of a thesis report: “Développement et caractérisation d'une puce a cellules pour le criblage d'agents toxiques” (Development and characterization of cell chip for screening toxic agents).

Moreover, the applicant filed on Jun. 23, 2009 a patent application FR 0954288 claiming advantageous dimensions of these microstructures, in particular with view to application to a liver cell culture.

The set of microstructures comprises:
bumps 111.4 with micrometric dimensions designated as “microbumps” in the following, and
walls 111.3 of micrometric dimensions designated as “microwalls” in the following,

By “overall surface area” of the bottom wall 111 is meant the surface area which the bottom wall 111 would have if the microstructures were projected onto the wall 111 in the vertical direction. This is the surface area of the rectangle formed by the wall 111

The microwalls 111.3 have arrow-shaped portions and straight portions and extend over the whole length of the bottom wall 111.

A microwall 111.3 defines microchambers 111.0 at its arrow-shaped portions in complementarity with another microwall 111.3, and microchannels 111.2 at its straight portions in complementarity with microbumps 111.4 as illustrated in FIG. 2.

Each microbump 111.4 defines two microchannels located on either side of the microbump, each in complementarity with a microwall.

The bottom wall 111 of the first chamber 110 thus comprises periodic lines in its longitudinal direction d1, over the whole of its width. Each line comprises an alternation of microchambers 111.0 and of microchannels 111.2, both lines being separated by a microwall 111.3.

As a non-limiting example, each line may comprise nine microchambers 111.0 and eight bumps 111.4—each corresponding to two microchannels 111.2—in alternation, the bottom wall 111 comprises a total of 15 lines.

Such a device has on the bottom wall 111 a geometry favorable to the development of cells, in particular as compared with planar culture devices such as Petri dishes. The microstructures allow organization of the cells in three dimensions provided that they are supplied with nutritious fluid containing elements required for development of the cells, in particular oxygen or glucose.

### Cell Culture in a Stagnating Fluid

For this purpose, the device according to the first aspect of the invention allows feeding of the cells without a circulation of nutritious fluid being necessary in the first chamber. Indeed, by placing stagnating fluid in the first chamber where the cells are brought to development, and by circulating a nutritious fluid in the second chamber, the elements required for development of the cells (in particular glucose) pass into the stagnating fluid, if the membrane 130 is selected suitably. In particular, it is possible to select a membrane for which the porosity is suitable for letting through glucose.

Thus, the device 100 according to the first aspect of the invention, finds a first application in the cultivation of cells. Indeed, a circulating nutritious fluid may carry away cells and break up the structures which is an obstacle to the development of cells and limits their activity. The device 100 described above gives the possibility of going beyond this difficulty by proposing a feeding solution without any circulation of fluid in direct contact with the cells.

However, the pressure of the fluid circulating in the second chamber may cause deformation of the membrane 130 which then moves nearer to the bottom wall 111. The membrane 130 is then a mechanical threat for the developing cells; it risks breaking up the structures of the cells and tearing them off the wall 111.

Now, the microstructures protect the cells from this harmful effect. Indeed, even if the membrane 130 would come into contact with the bottom wall 111, it would be in contact with the microwalls 111.3 and the microbumps 111.4, the cells being always able to develop in the microchambers 111.0 and the microchannels 111.2. Thus, the microchambers and the microchannels form a structure not only favorable for development of the cells, but also protected in the case when the membrane 130 deforms as far as the bottom wall 111.

The thereby described device 100 may be used in a filtration system according to the second aspect of the invention as this is illustrated in FIG. 4. The system further comprises a device 100, a fluid circuit 300 comprising circulation piping 310 provided with a circulation means 320. By <<piping>> is meant a set of one or more pipes. These pipes may be flexible or rigid, and consist of any suitable material known to one skilled in the art. The fluid circuit 300 is connected to the first and second openings 122, 123 in the second chamber 120, via respective passages 151 and 152 in the second block 102.

The circulation means 320 is preferentially connected to a supply 340 of nutritious medium, and may comprise a liquid pump, a peristaltic pump, a set of valves, for example solenoid valves, or any other suitable means know to one skilled in the art.

Further, the circuit 300 preferentially comprises a discharge conduit 350 for the nutritious fluid after its passing into the second chamber 120.

Such a system according to the second aspect of the invention is not limited to this illustration in which the circuit 300 is open, and in particular extends to any system in which the circuit 300 is closed and optionally comprises a means for regenerating nutritious fluid.

### Fluid Circulation in the First Chamber

Moreover, the device 100 according to the first aspect of the invention is not limited to the description made of it up to now. Alternatively, provision is made for the possibility of also circulating a fluid in the first chamber 110.

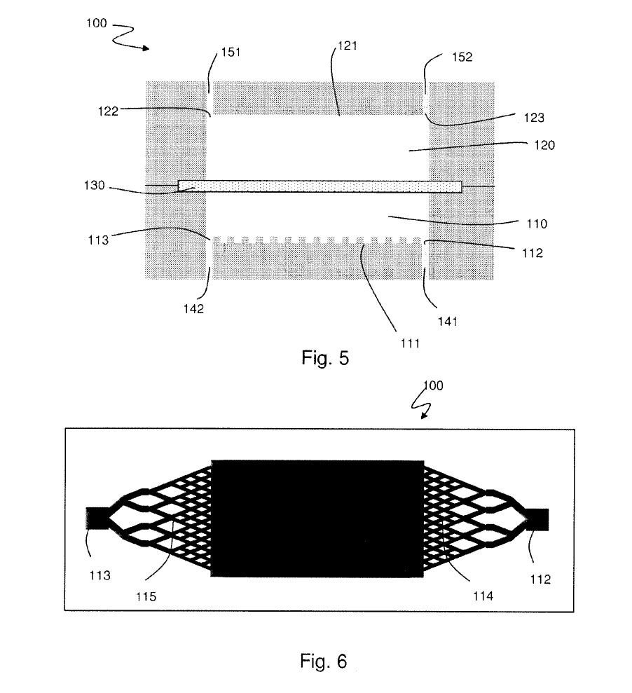

For this purpose, the device 100 according to the first aspect of the invention, further comprises a fluid inlet 112 and a fluid outlet 113 as illustrated in FIG. 5.

The fluid inlet 112 is connected to at least one portion of the microchannels 111.2 via an inlet network 114. The inlet network 114 comprises successive branches for supplying each of the lines of the bottom wall 111 of the first chamber from the fluid inlet 112.

The fluid is intended to circulate at the microchannels 111.2 and the microchambers 111.0, and above the microstructures in the first chamber 110, for example for feeding developing cells.

The fluid outlet 113 is connected to at least one portion of the microchannels via an outlet network 115. The outlet network 115 comprises successive confluence points for connecting each of the lines of the bottom wall 111 of the chamber 110 to the fluid outlet 113. The inlet 114 and outlet 115 networks of the bottom wall are illustrated in FIG. 6, in this advantageous alternative of the invention.

Thus, the microchannels 111.2 form a network connecting the fluid inlet 21 to each microchamber 111.0—via the inlet network 114—and each microchamber to the fluid outlet 113—via the outlet network 115. The microchambers 111.0 preferentially comprise an inlet area 115 and an outlet area 116 for allowing circulation of the fluid substantially in the direction d1—the longitudinal direction of the wall 111.

Many applications of the device according to this advantageous alternative of the first aspect of the invention are contemplated.

For example, provision may be made for circulating in the chamber 110 a fluid containing molecules to be tested on the cells. This application is particularly of interest in the screening of toxic substances for human beings: a human cell tissue is cultivated, fed through the membrane with a nutritious fluid circulating in a chamber 120 and directly exposed to test molecules in the chamber 110.

Moreover, is possible to circulate in the chamber 110 a discharge fluid for continuously removing cell secretions.

Another possible application is mechanical stimulation of the cells. Certain cells, such as endothelial cells are naturally subject to flow conditions like blood. These cells are naturally activated by friction, which may be reproduced by circulation of the fluid in the first chamber 110.

### Dimensions of the Microstructures

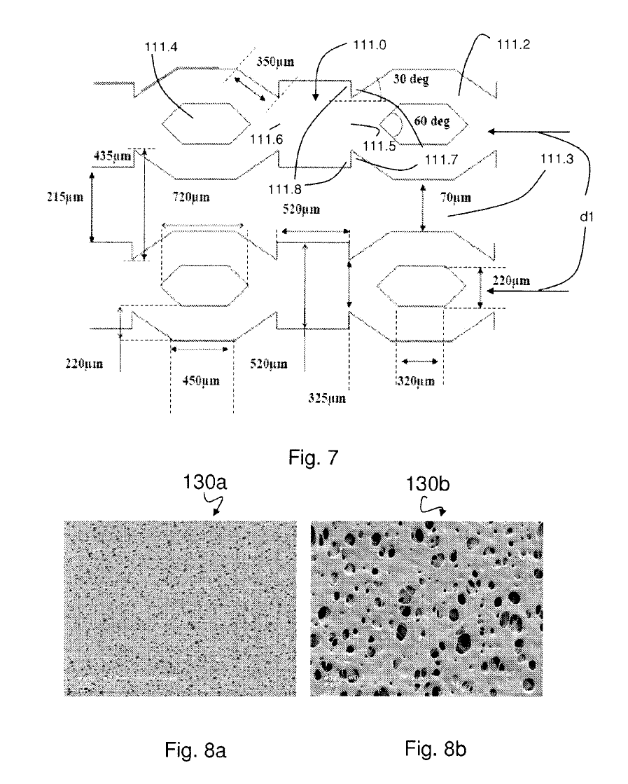

Advantageous dimensions of the microstructures will now be described according to a possible embodiment of the first aspect of the invention with reference to FIG. 7.

In this advantageous alternative, the microchambers 111.0 have a length dimension and a width dimension relatively to the direction d1, each comprised between 500 μm and 550 μm, preferentially 520 μm. Thus, the dimensions of the microchambers 111.0 of the filtration device 100 are advantageously 520 μm×520 μm×100 μm.

These dimensions are particularly favorable for developing liver cells in the microchambers 111.0.

Still advantageously, the microchannels 111.2 have relatively to the direction d1, a length dimension comprised between 700 μm and 750 μm, preferentially 720 μm, and a width dimension comprised between 200 μm and 250 μm, preferentially 220 μm. Thus, the dimensions of the microchannels 111.2 of the device 100 are advantageously 720 μm×220 μm×100 μm.

The thereby dimensioned microchannels 111.2 facilitate development of liver cells; in particular they allow migration of a piece of a liver organ through the network of microchannels.

Moreover, in FIG. 6, several other characteristic dimensions of the microwalls 111.3, microbumps 111.4 and microchannels 111.2 are illustrated for a possible embodiment of the filtration device 100 of the first aspect of the invention.

According to an advantageous alternative, the microwalls 111.3 comprise angled areas 111.7 on either side of the inlet area 111.5 and of the outlet area 111.6 of at least one chamber 111.0, as illustrated in FIGS. 4 and 6.

These angled areas 111.7 have a width dimension relatively to the direction d1 advantageously comprised between 100 μm and 120 μm, preferentially 110 μm. In particular, they have an edge transverse to the fluid circulation direction d1. The angled areas 111.7 define, relatively to the direction d1, partly protected areas on either side of the inlet area 111.5 of said chamber 110, i.e. areas where the circulation of the fluid is suddenly slowed down.

Thus, the cells developing in such partly protected areas are unlikely to be carried off by the fluid circulating in the microchamber 111.0.

Naturally, the extent of an area where the cells are protected from being carried off by the fluid depends on circulation conditions, in particular on the flow rate and on the shearing.

Nevertheless, the partly protected areas according to this advantageous alternative of the first aspect of the invention have an edge transverse to the direction d1 with a width of at least 100 μm.

They thereby allow aggregation of the cells at corners 111.8 of the microchamber 111.0 positioned transversely on either side and downstream from the inlet area 111.5 relative to the direction d1. In particular, liver cells may aggregate as a spheroid of a large diameter of the order of 100 μm, a favorable shape for good cell activity.

Generally, the network of microchannels allows the cells to develop and to aggregate in three-dimensional structures at development areas, i.e.:
the portion of the bottom wall 111 at the microchambers 111.0,
the portion of the bottom wall 111 at the microchannels 111.2,
the side walls of the microwalls 111.3, and
the side walls of the microbumps 111.4.

By culture surface area is designated the whole of these development areas. Advantageously, the ratio between the culture surface area and the overall surface area of the bottom wall 111 is comprised between 90% and 110%. It is preferentially equal to 100% to within an accuracy of 1%.

As a non-limiting numerical example, the culture surface area may be broken down in the following way (for an overall surface area of the bottom wall of 149 mm²):
surface area of the bottom wall at the microchambers 111.0 and at the microchannels 111.2: 96.5 mm²,
surface area of the side walls of the microwalls 111.3: 36.5 mm²
surface area of the side walls of the microbumps 111.4: 18 mm².

The culture surface area is therefore 151 mm².

The ratio of the culture surface area over the overall surface area of the bottom wall is therefore, in this example 101%.

Thus, the microstructures on the bottom wall 111 almost do not modify the surface area available for the culture relatively to the overall surface area of the bottom wall 111, while allowing three-dimensional development.

Further, cells may also develop on the upper surfaces of the microwalls 111.3 and of the microbumps 111.4, although such areas are not particularly favorable for three-dimensional development.

The culture surface area defined earlier to which are added the upper surface areas of the microwalls 111.3 and of the microbumps 111.4 is then called a <<total culture surface area>>.

In the previous numerical example, these upper surface areas are 52.5 mm² and the total culture surface area is 203.5 mm² and the ratio between the total culture surface area and the overall surface area of the bottom wall is 137%.

Still advantageously, the culture chamber 10 has a volume and the ratio R2 between the total culture surface area and the volume of the culture chamber 10 is comprised between 4 mm⁻¹ and 6 mm⁻¹.

If the volume is too small relatively to the total culture surface area (R2>6 mm⁻¹), the cells risk being confined and not having sufficient nutrients distributed by the fluid, which is harmful to their development.

Moreover, a too large volume relatively to the total culture surface area (R2<4 mm⁻¹), is uninteresting; it is actually preferable to have miniaturized devices.

By volume of the first chamber 110 is meant the available volume for the passage of the fluid; the volume occupied by the microwalls 111.3 and the microbumps 111.4 is therefore excluded.

By taking up the values of the preceding numerical example, and for a chamber height of 0.2 mm, the volume of the culture chamber 110 may be determined: 149×0.2−52.5×0.1=24.55 mm³.

The ratio between the total culture surface area and the volume of the first chamber 110 is then 203.5 mm²/24.55 mm³=8.29 mm⁻¹.

Advantageously, the upper surface 121 of the second chamber 120 also has microstructures. These microstructures may have all the advantageous alternatives of the microstructures detailed up to now relating to the bottom wall 111 of the first chamber 110. The microstructures may be of identical dimensions on the lower 111 and upper 121 walls, or else of different dimensions. This alternative is particularly of interest for an application with a view to cultivating cells in both chambers.

Contemplated Membranes within the Scope of the Invention

Different characteristics of the membrane 120 contemplated within the scope of the invention will now be described in more detail.

The membrane 130 is preferably in a flexible material and may thus be slightly deformable depending on the pressure prevailing in each of the chambers 110, 120. As this was seen earlier, the microstructures prevent the membrane 130 from adhering to the walls and protect the culture cells from a possible contact with the membrane.

The membrane 130 may be hydrophilic or hydrophobic, and will be selected depending on the targeted application (dialysis, cell culture, . . . ).

A membrane is said to be hydrophilic when there is an interaction of the terminal groups with water through a hydrogen bond. Hydrophilic membranes like cellulose membranes have good diffusion and as they have a low adsorption of the proteins, they have good convection; on the other hand the biocompatibility is poor. They are used in dialysis for letting through water and very small solutes.

Hydrophobic membranes, like synthetic membranes, have a lower diffusion but a higher ultrafiltration coefficient because of their porous structure which counterbalances the negative effect of the adsorption of proteins; the latter is at the origin of better biocompatibility. They are often used in hemodiafiltration. The biocompatibility of the membranes leads to many applications of these membranes with cell culture.

The membranes may be microstructured with microstructures of the micropore type (see FIGS. 8a and 8b) but also with micropores which may also assume the shape of microchannels or micropillars or microgeometries, for example geometries as illustrated in FIG. 7.

Advantageously, the membrane 130 may be hydrophilic, which limits adhesion of bacteria and of proteins and reduces the resistance to the passing of a fluid in the pores of the membrane 130.

Alternatively, the membrane 130 may be hydrophobic, which limits adhesion of bacteria, facilitates adhesion of proteins and increases the resistance to the passing of fluid in the pores.

Different porosities may be used for the membrane 130 depending on the size of the elements to be filtered. According to an advantageous alternative, the membrane 130 is preferably a barrier membrane, i.e. it only allows diffusion of small molecules or gases and prevents the passing of fluids from one chamber 110, 120 to the other chamber 120, 110. FIGS. 8a and 8b show images taken by electron microscopy of two exemplary filtration membranes 130a, 130b in polyethersulfone with a respective porosity of 40,000 Da and 500,000 Da.

According to a possible embodiment of the invention, the membrane 130 is selected so as to allow cultivation of cells on the membrane 130. In particular, provision is made for advantageously cultivating model cells of biological barriers (for example the intestinal barrier or a brain barrier) on the membrane, such as MDCK or Caco-2 cells

Preferentially, but in a non limiting way, the membrane 130 has a surface area of the order of 1 cm².

### Separation of the Blocks and of the Membrane

Advantageously, the filtration device 100 according to the first aspect of the invention comprises a holding means 160 for holding together the first block 101, the membrane 130 and the second block 102 in this order.

The means 160 has a locked configuration in which the first block 101 and the membrane 130 are held firmly together, on the one hand, the membrane 130 and the second block are held firmly together on the other hand, and an unlocked configuration, in which the block 101 and the membrane 130 may be separated from each other and/or in which the membrane 130 and the second block 102 may be separated from each other.

Further, the holding means 160 may be switched from the locked configuration to the unlocked configuration and vice versa, for example by action of a user.

The means 160 may comprise screws crossing the first block 101, the membrane 130 and the second block 102 over the whole of their height, as illustrated in FIG. 1. The means 160 may also comprise stops, a vice or any other suitable means known to one skilled in the art.

Such a holding means 160 has several advantages. It allows the separation of the device 100 into its constituents and the possibility of then rebuilding it. Thus, it is possible to access the chambers 110 and 120 as well as the membrane 130, without making the device 100 unusable.

This is most particularly useful for cleaning the chambers of the molecules to be filtered which are adsorbed on the surfaces and for sterilizing them, for example, with an autoclave or by any other suitable cleaning method known to one skilled in the art.

Moreover, the membrane 130 may be recovered for subjecting it to analysis, such as measurements of transmembrane electric resistance, recovery of the membrane in order to produce fluorescent markings on the cells, impedance analysis, or any other useful analysis known to one skilled in the art. In the case of a membrane receiving a cell culture, the means 160 also provides the possibility of directly accessing the cell culture while avoiding the discharge of this culture from the device 100 with a fluid which would destroy the culture structure.

Further, the membrane may be replaced with a new membrane for repetitive experiments without requiring cleaning. It is thus possible to repeat an experiment by changing the membrane 130 while keeping intact the cell contents of the chambers 110, 120.

Conversely, a same membrane 130 may be transplanted from one device 100 to another and be the subject of experiments with chambers 110, 120 with a geometry of different microstructures. This may be useful for characterizing the effect of the microstructures on the filtration or on a cell culture on the membrane 130.

A Filtration System with Two Fluid Circuits

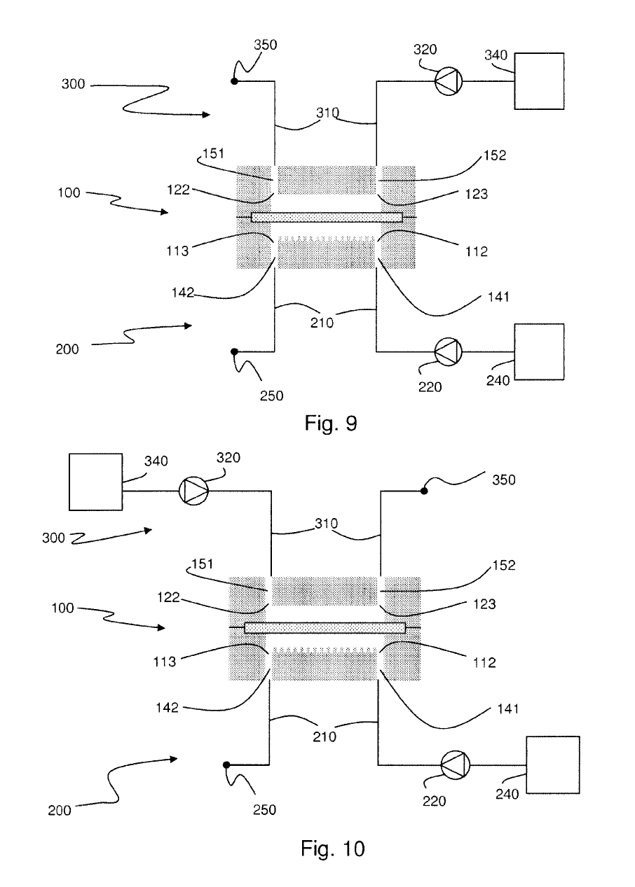

A filtration system according to several possible embodiments of the second aspect of the invention will now be described with reference to FIGS. 9 to 11.

In the alternatives considered below, the device 100 integrated into the filtration system comprises a fluid inlet 112 and a fluid outlet 113 in the first chamber 110.

The system comprises a fluid circuit 200 connected to the first chamber 110, similar to the circuit 300 connected to the second chamber 120 which has already been described above. The fluid circuit 200 comprises circulation piping 210 provided with a circulation means 220 and is connected to the inlets 112 and outlet 113 in the first chamber 110. The fluid circuit 200 is connected to the fluid inlets 112 and outlet 113 in the first chamber 110 via respective passages 141 and 142 in the first block 101.

The circulation means 320 is preferentially connected to a fluid supply 240 and may comprise a liquid pump, a peristaltic pump, a set of valves, for example solenoid valves, or any other suitable means known to one skilled in the art.

Further, the circuit 200 preferentially comprises a discharge conduit 250 for the fluid after passing in the first chamber 110.

Such a system according to the second aspect of the invention is not limited to this illustration in which the circuit 200 is open, and in particular extends to any system in which the circuit 200 is closed and optionally comprises a means for regenerating the fluid.

Moreover, the fluids circulating in the circuits 200 and/or 300 are advantageously temperature-controlled. For example, the supplies 240 and/or 340 may be arranged in thermostated baths (not shown). Alternatively, or cumulatively, any other means for controlling the temperature of the circuits 200 and/or 300 known to one skilled in the art may be contemplated.

In the alternative embodiment illustrated in FIG. 9 the fluids contained in the lower 110 and upper 120 chambers circulate as co-currents. According to this configuration, the first opening 122 is a fluid outlet of the second chamber 120 and the second opening 123 is a fluid inlet in the second chamber 120.

Moreover, in FIG. 10, an alternative embodiment is illustrated in which the fluids contained in the lower 110 and upper 120 chambers circulate as countercurrents. According to this configuration, the first opening 120 is a fluid inlet in the second chamber 120 and the second opening 123 is a fluid outlet of the second chamber 120.

The systems described by FIGS. 9 and 10 notably find application in the field of hemodialysis. Blood to be treated may circulate in the second chamber 120 and be cleared of certain components ordinarily eliminated by a functional kidney, by filtration through the membrane 130. A dialysis fluid contained in the first chamber 110 may then discharge the filtered elements and ensure provision of glucose for the patient.

Such systems may also be used for characterizing a filtration membrane 130. For example, it is possible to evaluate a diffusion coefficient of a molecule for a given membrane 130 from a physical model and measurements of concentration of the molecule in the first chamber 110 and/or the second chamber 120 versus time. The co-current system may be used for calibrating the model and estimating the diffiusion coefficient, and the countercurrent system may be used for checking the diffusion coefficient or vice versa.

In particular, the applicant modeled the time-dependent change of the concentration in the first chamber 110, in the case when a fluid to be filtered circulates in the second chamber 120, with the following equations:
in the case of a co-current flow (like in FIG. 9):

$${C}_{1} ( t ) = {C}_{1 ∞} + ( {C}_{1 o} - {C}_{∞} ) exp [ - \frac{{LbD}_{m}}{δ} ( \frac{{V}_{1} + {V}_{2}}{{V}_{1} {V}_{2}} ) t ]$$

in the case of a counter-current flow (like in FIG. 10):

$${C}_{1} ( t ) = {C}_{1 ∞} + ( {C}_{1 o} - {C}_{∞} ) exp [ - {Q}_{f} ( 1 + \frac{2}{Pe} ) ( \frac{{V}_{1} + {V}_{2}}{{V}_{1} {V}_{2}} ) t ]$$

wherein,

$$Pe = \frac{{Q}_{f}}{( bl / 2 )} \frac{δ}{{D}_{m}} ,$$

and
C₁(t) represents the concentration in the first chamber 110 as a function of time t,
L represents the (common) length of the first and second chambers 110, 120,
b represents the (common) width of the first and second chambers 110, 120,
Q_{f} represents the filtration flow rate,
V₁ represents the volume of the first chamber 110,
V₂ represents the volume of the second chamber 120,
C₁ₒ represents the initial concentration in the first chamber 110,
C_{1∞} represents the equilibrium concentration in the first chamber 110,
Dₘ represents the diffusion coefficient of the molecule to be filtered for the membrane 130, and
δ represents the thickness of the membrane.

By means of these models, it is possible to determine the values of the diffusion coefficient Dₘ of the molecules tested for a given membrane 130. For this, for example it is possible to operate in the co-current mode and measure the disappearance of the molecule in the second chamber 120 (and therefore its appearance in the first chamber 110).

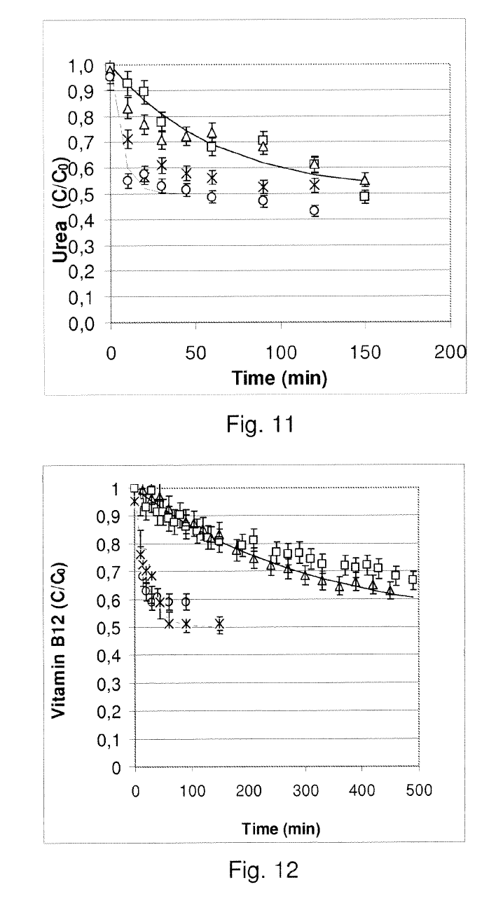

In FIGS. 11 and 12 the change in the concentration-over-initial-concentration-in-the-second-chamber 120 ratio is illustrated versus time for given experimental conditions, for urea (FIG. 11) and vitamin B12 (FIG. 12) respectively, with several types of membrane. Moreover, the applicant has also determined this ratio for albumin (graph not shown).

The experimental results are illustrated, for an experiment with a membrane of high porosity (of the 8 F type) by icons □, Δ, and for an experiment with a membrane of low porosity (of the 1FPH type) by icons: *, ∘. A regression of the analytic model is then made for example with a minimization by least squares, in which it is possible to obtain experimentally the diffusion coefficients in (m²/s) listed in the following table:

| | Urea | Vitamin B12 | Albumin |
|---|---|---|---|
| 1 FPH | 2 × 10⁻¹² m²/s | 4 × 10⁻¹³ m²/s | 9 × 10⁻¹⁴ m²/s |
| 8 F | 2 × 10⁻¹¹ m²/s | 6 × 10⁻¹² m²/s | 2 × 10⁻¹³ m²/s |

Thus, the larger the molecule, the smaller is the diffusion coefficient. Further, the more porous the membrane, the larger is the diffusion coefficient.

The validity of the diffusion coefficient was then proved by means of the countercurrent model.

Thus, it is possible to characterize a membrane with a microsystem requiring a membrane surface area of the order of one cm². If necessary, the estimated characteristics may be extrapolated on membranes of larger size.

### Pressure Control in the Chambers

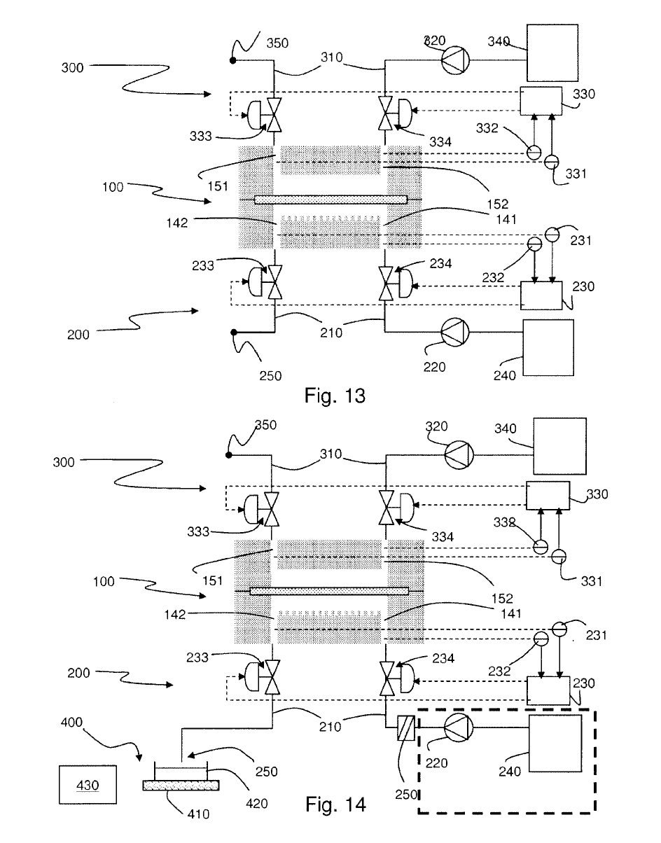

With reference to FIG. 13, the circulation circuit 200 advantageously comprises a fluidic pressure control means 230 in the first chamber 110, or else the circulation circuit 300 advantageously comprises a fluidic pressure control means 330 in the second chamber 120, or else both circuits 200, 300 each comprise a pressure control means 230, 330 in their associated chamber 110, 120.

Preferentially, but not as a limitation, the control means 230 (330 respectively) comprises pressure sensors 231, 232 (331, 332 respectively), for detecting pressure of the fluid in the passages 141, 142 in the first block 101 (151, 152 in the second block 102, respectively) or at the fluid inlet and outlet 112, 113 (at the first and second openings 122, 123, respectively). The means 230 (330 respectively) further comprises actuators 233, 234 (333, 334, respectively) positioned on the piping 210 (310 respectively) in order to modify the pressure at the inlet and outlet of the fluid in the first chamber 110 (in the second chamber 120, respectively). These actuators may for example be solenoid valves controlled by a unit (not shown) for processing data from pressure sensors or any other suitable means known to one skilled in the art.

The pressure controls may be carried out by the processing unit of each control means 230, 330 by stabilizing the pressure around a fixed or variable set value for example by applying a proportional controller, a proportional-integral controller, an open loop (in which case the pressure sensors are not used) or any other suitable control loop known to one skilled in the art of system control.

The control means 230, 330 of the circuits 200, 300 may be similar or different according to the needs of the considered application for the filtration system according to the second aspect of the invention.

They prove to be very useful for monitoring certain parameters during the filtration such as the discard rate or the diffusion coefficient. The conditions of flow rates and transmembrane flow may thus be closely controlled.

The pressure difference may be maintained at a determined value so that the membrane will not adhere onto the walls.

Moreover, the means 230, 330 allow control of the conditions of flow rates in the chambers 110, 120 and the transmembrane flow during filtration. This allows control of the parameters of the filtration for a given solute, such as the discard rate, i.e. the percentage of dissolved material retained by the membrane, and the diffusion coefficient of the solute through the membrane 130.

Thus, the means 230, 330 give the possibility of proceeding with repetitive experiments under the same conditions of flow rate and of transmembrane flow.

A particularly interesting application of this advantageous effect is to test different membranes under similar experimental conditions, which allows characterization of the properties of the membranes, for example the water slope—i.e. the hydraulic permeability of the membranes to pure water—for example via the experiment illustrated in FIG. 14.

In this experiment, the fluid circulating in the second chamber 120 is water. There is no cell culture in the first chamber 110, a first chamber 110 which is further isolated from the supply 240 and from the circulation means 220, by a means 250 for short-circuiting the first circuit 200. Alternatively, it may be considered in this example that the first circuit 200 does not comprise any supply 240 nor any circulation means 220, in which case the short-circuiting means 250 is unnecessary. In order to illustrate the latter, the supply 240 and the means 220 are surrounded by a rectangle in dotted lines in FIG. 14.

Further, the discharge conduit 350 of the second circuit 300 is blocked for water (for example a closed tap). Thus, the water circulating in the second chamber through the second opening 152 can only flow out of the device 100 through the passage 140 of the first block 101, which passage 142 is connected to the discharge conduit 250 of the first circuit 200 through the piping 210.

Moreover, the system according to this alternative of the second aspect of the invention is associated with a device 400 for measuring the mass of water, comprising a container 410 connected to the discharge conduit 250, scales 420 and a processing unit 430 interacting with the scales 420 and intended for determining the mass of water contained in the container 410 versus time. Moreover, the processing unit receives information from means 230, 330 for controlling pressure in both circuits 200, 300.

Thus, the device 400 is capable of evaluating the mass of water filtering through the membrane 130 versus time and the transmembrane pressure, which allows determination of the water slope of the membrane 130.

The experiment was conducted by the applicant on the membranes 130a and 130b illustrated in FIGS. 8a and 8b respectively. The determined water slopes are 8 mL/(min. bar. cm³) and 80 mL/(min. bar. cm³), respectively.

Another application is to measure the pressure drops in the first chamber 110 (and in the second chamber 120 if it receives a cell culture), which pressure drops give an indication on the variations of the number of culture cells in the chamber.

### Devices Connected in Series

Finally, it is possible to contemplate the putting of several filtration devices 100 in series thereby allowing the operating steps of a kidney to be reproduced entirely, each device reproducing a particular function. More generally, several devices 100 may be used in series for reproducing the interactions between a fluid and various cell tissues in the body.

### CONCLUSION

The invention has many advantages. The system according to the second aspect of the invention allows control of filtration as compared with larger systems. The circuits for the fluids do not experience turbulence and only very little edge effects, whether this be in the piping as in the chambers of the device according to the first aspect of the invention. With the means for controlling pressure and the membrane, it is possible to maintain uniform parameters (pressure, temperature, composition and concentration of the fluids) so that the observed results may easily be extrapolated to systems with similar parameters. Thus, the invention is a great step towards in vitro reproduction of phenomena of the human or animal body, as well as towards making artificial organs.

1. A filtration device (100) characterized in that it comprises:
a first block (101) having a cavity forming a first chamber (110) including a bottom wall (111) having a set of microstructures comprising microwalls (111.3) and microbumps (111.4), the set of microstructures defining on the bottom wall (111) of the culture chamber, microchambers (111.0) and microchannels (111.2),
a second block (102) having a cavity forming a second chamber (120), and
a filtration membrane (130),
the first block (101), the membrane (130) and the second block between (102) being laid out so that the membrane (130) is located between the first chamber (110) and the second chamber (120) adjacent to each of the first and second chambers (110), (120),
a first opening (122) and a second opening (123), for letting through a fluid into the second chamber (120) separated from the first chamber (110) by the membrane (130).

2. The device (100) according to the preceding claim, further comprising:
a fluid inlet (112) in the first chamber (110) connected to at least one portion of the microchannels (111.2),
a fluid outlet (113) in the first chamber (110) connected to at least one portion of the microchannels (111.2),
the microchannels forming a network connecting the fluid inlet (112) to each microchamber (111.0) and each microchamber (111.0) to the fluid outlet (113), so as to allow circulation of fluid in the microchambers (111.0) of the first chamber (110).

3. The filtration device (100) according to one of the preceding claims, wherein the microchambers (111.0) have a length dimension and a width dimension relatively to a circulation direction of the fluid each comprised between 500 μm and 550 μm.

4. The filtration device (100) according to one of the preceding claims, wherein the microchannels (111.2) have a length dimension relative to a circulation direction of the fluid comprised between 700 μm and 750 μm and a width dimension relative to a circulation direction of the fluid comprised between 200 μm and 250 μm.

5. The filtration device (100) according to one of the preceding claims, wherein the microchambers (111.0) comprise an inlet area (111.5) and an outlet area (111.6), the microwalls (111.3) comprising angled areas (117) on either side of the inlet area (115) and of the outlet area (116) of at least one chamber (110), the angled areas (117) having a width dimension relative to a circulation direction of the fluid comprised between 100 μm and 120 μm so as to define on either side of the inlet area (111.5) of said chamber (111.0), partly protected areas.

6. The filtration device (100) according to one of the preceding claims, wherein the first chamber (110) has a culture surface area in a ratio with an overall surface area of the bottom wall (111) comprised between 90% and 110%.

7. The filtration device (100) according to one of the preceding claims, wherein the first chamber (110) has a culture surface area in a ratio with an available overall volume of fluid of the first chamber (110) comprised between 4/mm and 6/mm.

8. The filtration device (100) according to one of the preceding claims, wherein the second chamber (120) comprises an upper wall (121) having a set of microstructures identical with that of the bottom wall (111) of the first chamber (110).

9. The filtration device (100) according to one of the preceding claims, wherein the membrane (130) is in a flexible material.

10. The filtration device (100) according to one of the preceding claims, wherein the membrane (130) is in a hydrophilic material.

11. The filtration device (100) according to one of claims 1 to 9 wherein the membrane (130) is in a hydrophobic material.

12. The filtration device (100) according to one of the preceding claims, wherein the membrane (130) is a barrier membrane.

13. The filtration device (100) according to one of the preceding claims, wherein the membrane (130) is in a suitable material for allowing cultivation of cells on the membrane (130).

14. The filtration device (100) according to one of the preceding claims, further comprising a holding means (160) having a locked configuration in which the first block (101) and the membrane (130) are held firmly together on the one hand, the membrane (130) and the second block are held firmly together on the other hand, and an unlocked configuration, in which the block (101) and the membrane (130) may be separated from each other and/or in which the membrane (130) and the second block (102) may be separated from each other, the holding means (160) being able to switch from the locked configuration to the unlocked configuration and vice versa.

15. A filtration system, characterized in that it comprises:
a filtration device (100) according to one of claims 1 to 14, and
a fluid circuit (300) comprising circulation piping (310) provided with a circulation means (320) and connected to the first and second openings (122, 123) in the second chamber (120).

16. The filtration system according to the preceding claim, wherein the filtration device (100) comprises
a fluid inlet (112) in the first chamber (110) connected to at least one portion of the microchannels (111.2),
a fluid outlet (113) in the first chamber (110) connected to at least one portion of the microchannels (111.2),
the microchannels forming a network connecting the fluid inlet (112) to each microchamber (111.0) and each microchamber (111.0) to the fluid outlet (113), so as to allow fluid circulation in the microchambers (111.0) of the first chamber (110),
the device further comprising a fluid circuit (200) comprising circulation piping (210) provided with a circulation means (220) and connected to the fluid inlet (112) and outlet (113) in the first chamber (110).

17. The filtration system according to one of claims 15 to 16, wherein the first circuit (200) further comprises a control means (230) for controlling a fluid pressure in the first chamber (110).

18. The filtration system according to one of claims 15 to 17 wherein the second circuit (300) further comprises a control means (330) for controlling a fluid pressure in the second chamber (120).

19. A filtration system characterized in that it comprises several filtration devices (100) according to one of claims 1 to 14, connected in series through circulation circuits.
